(19) United States
(12) Patent Application Publication
(10) Pub. No.: US 2023/0000939 A1
(43) Pub. Date: Jan. 5, 2023

(54) PRODUCTION METHOD FOR PURIFIED SALACIA GENUS PLANT EXTRACT, AND PURIFIED SALACIA GENUS PLANT EXTRACT

(71) Applicants: FUJIFILM Corporation, Tokyo (JP); FarFavour Enterprises Ltd., Hangzhou (CN)

(72) Inventors: Meng ZHENG, Hangzhou (CN); Fuli NIE, Hangzhou (CN); Quanmin MA, Hangzhou (CN)

(73) Assignees: FUJIFILM Corporation, Tokyo (JP); FarFavour Enterprises Ltd., Hangzhou (CN)

(21) Appl. No.: 17/941,956

(22) Filed: Sep. 9, 2022

Related U.S. Application Data
(63) Continuation of application No. PCT/JP2021/009408, filed on Mar. 10, 2021.

(30) Foreign Application Priority Data
Mar. 11, 2020 (JP) 2020-041603

(51) Int. Cl. A61K 36/37

(52) U.S. Cl. CPC A61K 36/37

(57) ABSTRACT

An object of the present invention is to provide a purified Salacia genus plant extract that has reduced bitter taste and/or reduced astringent taste, in which the content of salacinol contained in the extract is not reduced during purification and the concentration of salacinol contained in the extract is maintained, and a production method therefor. According to the present invention, there are provided a purified Salacia genus plant extract in which the content of epicatechin and the content of epigallocatechin are each less than 0.001% by mass with respect to the total amount of the purified Salacia genus plant extract; and a production method for a purified Salacia genus plant extract, which includes an extraction step of passing a Salacia genus plant-containing raw material extract containing at least one of a Salacia genus plant, a Salacia genus plant extract, or a Salacia genus plant ground product, through a column packed with a styrene-based synthetic resin.

### CROSS-REFERENCE TO RELATED APPLICATIONS

This application is a Continuation of PCT International Application No. PCT/JP2021/009408 filed on Mar. 10, 2021, which claims priority under 35 U.S.C § 119(a) to Japanese Patent Application No. 2020-041603 filed on Mar. 11, 2020. Each of the above application(s) is hereby expressly incorporated by reference, in its entirety, into the present application.

### BACKGROUND OF THE INVENTION

### 1. Field of the Invention

The present invention relates to a production method for purified Salacia genus plant extract, and a purified Salacia genus plant extract.

### 2. Description of the Related Art

Roots and stems of Salacia genus plants have been utilized as natural drugs in the traditional medicine Ayurveda of India and Sri Lanka. In Sri Lanka, it has been passed down that the root bark of Salacia reticulata is effective for the treatment of rheumatism, gonorrhea, and skin diseases, and that the above-described root bark is used for the treatment of early stage diabetes mellitus.

In recent years, it has been reported that a Salacia genus plant extract has an effect of inhibiting a rise in the blood glucose level by a sugar absorption suppressing action based on α-glucosidase activity inhibition (for example, FOOD Style 21, Vol. 6, No. 5, pp. 72-78), and a Salacia genus plant extract includes polyphenols (and mangiferin, which is xanthene glycoside) (for example, YAKUGAKU ZASSHI, 121 (5), pp. 371-378). The described polyphenols are perceived as bitter taste, astringent taste, astringency, harsh taste, and odd taste, and they may give an unpleasant feeling to the consumers when ingested as food products.

By the way, examples of the substance containing polyphenols, similarly to the Salacia genus plant extract, include a tea extract. JP1997-220055A (JP-H9-220055A) discloses a production method for a tea beverage, characterized by bringing a tea extract liquid containing tannins and amino acids into contact with a polyvinylpolypyrrolidone resin to adsorb and remove the tannins in the tea extract liquid, thereby setting an amino acids/tannins ratio to 0.2 to 3.0. In addition, JP4336192B discloses a production method for catechins that have reduced fragrance and reduced bitter taste, which is characterized by passing an aqueous solution containing catechin extracted from tea leaves through a column packed with a synthetic adsorbent which is a copolymer of styrene and divinylbenzene and treating it to collect a non-adsorbed part thereof.

### SUMMARY OF THE INVENTION

Until now, there has been no report on a method of producing a Salacia genus plant extract suitable for the use application on oral ingestion, which does not require processing into a pharmaceutical preparation or a food product, by using a resin column for the intended purpose of reducing bitter taste and/or astringent taste. In a case of purifying a Salacia genus plant extract, attention should be paid to the recovery rate of the active ingredient salacinol of the Salacia genus plant extract and the concentration thereof so that it is not decreased.

A decrease in the recovery rate of the active ingredient salacinol due to purification is not preferable from the viewpoint of the production cost. Since a decrease in the concentration of the active ingredient salacinol leads to a reduction in the activity of the Salacia genus plant extract, the amount of one-time ingestion should be increased, and thus, there is a problem that the burden on the ingesting person may increase. Furthermore, in order to obtain an extract that can be directly ingested orally without being subjected to processing into a pharmaceutical preparation, a food product, or the like, it is important to produce an extract that has reduced bitter taste and/or reduced astringent taste and that can be easily ingested.

An object of the present invention is to provide a purified Salacia genus plant extract that has reduced bitter taste and/or reduced astringent taste, in which the content of salacinol contained in the extract is not reduced during purification and the concentration of salacinol contained in the extract is maintained, and a production method therefor.

In general, the raw material for obtaining a purified Salacia genus plant extract is not subjected to a purification treatment since the recovery rate is easily decreased. However, as a result of diligent studies to achieve the above object, the inventors of the present invention have found that the above problems can be solved by an extraction step of passing a Salacia genus plant-containing raw material extract consisting of at least one of a Salacia genus plant, a Salacia genus plant extract, or a Salacia genus plant ground product, through a column packed with a styrene-based synthetic resin.

According to the present invention, the following inventions are provided.

<1> A purified Salacia genus plant extract, in which a content of epicatechin and a content of epigallocatechin are each less than 0.001% by mass with respect to a total amount of the purified Salacia genus plant extract.

<2> The purified Salacia genus plant extract according to <1>, in which a content of mangiferin is less than 0.001% by mass with respect to the total amount of the purified Salacia genus plant extract.

<3> The purified Salacia genus plant extract according to <1> or <2>, in which in a case where a taste sensor that uses an artificial lipid membrane responding to bitter taste is immersed in a 0.3 mmol/L tartaric acid solution containing 30 mmol/L potassium chloride to acquire a membrane potential Vr, and the taste sensor is immersed in a sample solution containing the purified Salacia genus plant extract to acquire a membrane potential Vs, a foretaste that is a value calculated from Vs−Vr is 7.00 or less.

<4> The purified Salacia genus plant extract according to any one of <1> to <3>, in which in a case where a taste sensor that uses an artificial lipid membrane responding to bitter taste or astringent taste is immersed in a 0.3 mmol/L tartaric acid solution containing 30 mmol/L potassium chloride to acquire a membrane potential Vr, the taste sensor is immersed in a sample solution containing the purified Salacia genus plant extract to acquire a membrane potential Vs, the taste sensor is washed with a 0.3 mmol/L tartaric acid solution containing 30 mmol/L potassium chloride, and the taste sensor is immersed again in a 0.3 mmol/L tartaric acid solution containing 30 mmol/L potassium chloride to acquire a membrane potential Vr′, an aftertaste is 0.6 or less, where the aftertaste is determined, as a value of change of membrane potential by adsorption, from a change in membrane potential which is the change represented by Vr′−Vr.

<5> The purified Salacia genus plant extract according to any one of <1> to <4>, in which the purified Salacia genus plant extract is obtained by a method including an extraction step of passing a Salacia genus plant-containing raw material extract containing at least one of a Salacia genus plant, a Salacia genus plant extract, or a Salacia genus plant ground product, through a column packed with a styrene-based synthetic resin.

<6> A production method for the purified Salacia genus plant extract according to any one of <1> to <4>, the production method comprising an extraction step of passing a Salacia genus plant-containing raw material extract containing at least one of a Salacia genus plant, a Salacia genus plant extract, or a Salacia genus plant ground product, through a column packed with a styrene-based synthetic resin.

<7> The production method for a purified Salacia genus plant extract according to <6>, in which the extraction step is carried out at a flow rate of SV=0.25 to 50 for liquid passage.

<8> The production method for a purified Salacia genus plant extract according to <6> or <7>, in which a ratio of a diameter to a height in the column is 1:1 to 1:12.

<9> The production method for a purified Salacia genus plant extract according to any one of <6> to <8>, in which the styrene-based synthetic resin has a specific surface area of 300 to 1,500 m²/g.

According to the present invention, it is possible to provide a purified Salacia genus plant extract that has reduced bitter taste and/or reduced astringent taste, in which the content of salacinol contained in the extract is not reduced during purification and the concentration of salacinol contained in the extract is maintained.

### DESCRIPTION OF THE PREFERRED EMBODIMENTS

Hereinafter, an example of the embodiments of the present invention will be described. However, the present invention is not intended to be limited to the following embodiments, and modifications can be applied as appropriate within the scope of the purpose of the invention. The numerical value range expressed using the symbol “˜” according to the present specification means a range including the numerical values described before and after the symbol “˜” as the minimum value and the maximum value, respectively.

With regard to a numerical value range described stepwise in the present specification, the upper limit or lower limit described in one numerical value range may be substituted for the upper limit or the lower limit of another numerical value range described stepwise. Furthermore, with regard to the numerical value ranges described in the present specification, the upper limit or the lower limit of the numerical value range may be substituted for the values disclosed in Examples.

According to the present specification, the term “step” means not only an independent step; however, in a case where a step cannot be clearly distinguished from other steps, as long as the predetermined purpose of the step is achieved, the step is included in the present term.

[Salacia Genus Plant-Containing Raw Material Extract]

Salacia genus plants are plants of the family Hippocrateaceae growing naturally mainly in Sri Lanka, India, and the South-East Asia region. Specific examples of a Salacia genus plant include one or more plants selected from Salacia reticulata, Salacia oblonga, Salacia prinoides, Salacia chinensis, Salacia latifolia, Salacia burunoniana, Salacia grandiflora, or Salacia macrosperma. The Salacia genus plant is preferably at least one plant selected from Salacia reticulata, Salacia oblonga, or Salacia chinensis.

The “Salacia genus plant-containing raw material extract” means components derived from a Salacia genus plant among the raw materials for obtaining a purified Salacia genus plant extract in the extraction step of passing a liquid through a column packed with a styrene-based synthetic resin, and other components included in the extract (for example, an extraction solvent used for preparing the raw material) are excluded.

As the Salacia genus plant to be used for the Salacia genus plant-containing raw material extract, it is sufficient to use at least one of a Salacia genus plant, a Salacia genus plant extract, or a Salacia genus plant ground product. As the Salacia genus plant, edible parts such as roots, stems, leaves, flowers, and fruits of Salacia genus plants can be directly used.

The extract of a Salacia genus plant and the ground product of a Salacia genus plant are used in the sense that they include an extract and/or ground product of edible parts such as roots, stems, leaves, flowers, and fruits of Salacia genus plants, and dried products of the extract and/or ground product. The dried product may be a dried powder (an essence powder). At the time of preparing the above-described extract and/or ground product of a Salacia genus plant, one or more kinds of sites of the Salacia genus plant may be used as a mixture. From the viewpoint of producing a purified Salacia genus plant extract efficiently, a Salacia genus plant extract (an essence) extracted from a site selected from roots and stems, or an essence powder obtainable by drying an essence is preferably used as the Salacia genus plant-containing raw material extract.

A dried powder (an essence powder) can be preferably obtained by extracting edible parts and the like of a Salacia genus plant by means of a solvent and drying an extract obtained as described above.

Examples of the solvent that is used for extraction include water, an alcohol, and a ketone, and a mixed solvent obtained by mixing two or more kinds of these may also be used.

Examples of the alcohol include methanol and ethanol, and ethanol is preferred.

Regarding the ketone, acetone, methyl ethyl ketone, cyclohexane, and the like are preferred.

Among those described above, water, an alcohol, a mixed solvent of water and an alcohol, or a mixed solvent of water and a ketone is preferred; water, an alcohol, or a mixed solvent of water and an alcohol is more preferred; and hot water at 50° C. to 98° C., ethanol, or a mixed solvent of water and ethanol is still more preferred.

The alcohol content in the mixed solvent of water and an alcohol is preferably 30% by mass to 90% by mass, and more preferably 40% by mass to 70% by mass.

The drying method used at the time of drying an extract to obtain a dried powder (an essence powder) is not particularly limited, and known drying methods, for example, methods such as spraying drying and freeze-drying may be mentioned. An excipient may be used in the drying method as appropriate.

[Extraction Step]

The production method for a purified Salacia genus plant extract according to the embodiment of the present invention includes an extraction step of passing a Salacia genus plant-containing raw material extract containing at least one of a Salacia genus plant, a Salacia genus plant extract, or a Salacia genus plant ground product, through a column packed with a styrene-based synthetic resin.

(Styrene-Based Synthetic Resin)

The styrene-based synthetic resin that is used in the present invention can be used without particular limitation on the kind and characteristics thereof. For example, it can be synthesized by suspension polymerization in an aqueous bath by the radical copolymerization of styrene and divinylbenzene.

A commercially available product can be used as the styrene-based synthetic resin, and examples thereof include, DIAION (registered trade name) HP10, HP20, HP30, HP40, SEPABEADS (registered trade name) SP850, SP700 (all trade names, manufactured by Mitsubishi Chemical Corporation, Ltd.); Amberlite (registered trade name) XAD (registered trade name) 4, 1180N, 2000 (all trade names, manufactured by ORGANO CORPORATION); Nandai D1, Nandai D2, Nandai D3, Nandai D4, Nandai D5, Nandai D6, Nandai D8, Nandai DS2, Nandai DS5, Nandai DM2, Nandai DM4 (all trade names, manufactured by Tianjin Nankai University Resin Co., Ltd.); D101, MD, DA (all trade names, manufactured by Tianjin Lanxiao Technology Co., Ltd.); D101, LX-11, LX-60, LSA-10, LX-28, AB-8, LX-38, LSA-7, LX-8, XDA-8, LX-17, XDA-7, LX-10G, LX-68, LX-68G, XDA-200B, D101C, LSA-21, XDA-6, LSA-10, LXA-8, LX-1, LX-68M, LX-60, LX-38C, LX-17, LSD001, LSI-010, XDA-8E, LSA-700, LSD762, LSA-700B, D941, LSC-AS (all trade names, Xian Sunresin New Materials Co., Ltd.); SD200, SD300, SD600, ZGDM-11, ZGDM-130, ZGCAD45, ZGDM-180 (all trade names, Zhejiang Lanxiao Business Co., Ltd.); D3520, D4006, D4020, H103, H107, H1020, X-5, NKA, NKA-II, NKA-9, S-8, AB-8 (all trade names, manufactured by Tianjin Hakko Resin Technology Co., Ltd.); D301, D113, D201, D001 (all trade names, Lanxiao Nandai Resin Co., Ltd.); and D001, D201, D113, D301 (all trade names, manufactured by Tianjin Basio Resin Technology Co., Ltd.).

The specific surface area of the styrene-based synthetic resin is preferably 300 to 1,500 m²/g and more preferably 600 to 1,000 m²/g from the viewpoint of increasing the recovery rate.

The average pore diameter of the styrene-based synthetic resin is preferably 100 to 400 nm and more preferably 150 to 350 nm from the viewpoint of obtaining a purified Salacia genus plant extract having an increased salacinol concentration.

Regarding the average pore diameter and specific surface area of the styrene-based synthetic resin, in a case where the styrene-based synthetic resin is a commercially available product, the values described in catalogs can be employed. Furthermore, regardless of being a commercially available product or not, in a case where these values are not clearly known, the values can be measured by the methods for measuring a specific surface area based on gas adsorption according to JIS Z 8830 (2013) and ISO 9277 (2010).

Regarding a method of carrying out contact with a styrene-based synthetic resin, the Salacia genus plant-containing raw material extract is passed through a column packed with a resin, preferably at a flow rate SV=0.25 to 50, more preferably at a flow rate SV=0.50 to 50, still more preferably at a flow rate SV=0.50 to 30, and particularly preferably at a flow rate SV=0.50 to 20, from the viewpoint of efficiently obtaining a purified Salacia genus plant extract. SV means a space velocity, and it is a unit that indicates how many times the resin volume per hour is passed.

The ratio of the diameter to the height in the column is preferably 1:1 to 1:12 and more preferably 1:3 to 1:10 from the viewpoint that the work efficiently proceeds.

The detailed mechanism is not certainly understood; however, it is conceived to be because the salacinol concentration can be further increased since the styrene-based synthetic resin selectively removes specific components contained in the Salacia genus plant extract.

(Extraction Solvent)

Regarding the extraction solvent, any solvent can be used without any particular limitations on the type, characteristics, and the like thereof, and water and/or an organic solvent can be used; however, from the viewpoint of the production cost, the extraction solvent is preferably water. The type of water is not particularly limited, and water can be appropriately selected from tap water, distilled water, synthetic water, natural water, and the like, and used.

Furthermore, in the case of using an organic solvent, for example, examples include alcohols such as ethanol and methanol; ketones such as acetone; and esters such as ethyl acetate. A hydrophilic organic solvent such as an alcohol or a ketone is preferable. In consideration of the use in pharmaceutical preparations or food products, an alcohol is more preferable, and ethanol is still more preferable.

The extraction solvent may be an extraction solvent contained in the Salacia genus plant-containing raw material extract described above. That is, the solvent used to prepare a Salacia genus plant extract (an essence) that serves as a raw material may be used directly as the extraction solvent.

(Temperature)

The temperature at the time of carrying out contact with the styrene-based synthetic resin is preferably 10° C. to 50° C. and more preferably 15° C. to 30° C. from the viewpoint of increasing the salacinol concentration of the purified Salacia genus plant extract to be obtained.

According to the production method according to the embodiment of the present invention, it is possible to increase the concentration of salacinol contained in the extract and reduce the bitter taste without reducing the content of salacinol contained in the extract during purification. As a result, a purified Salacia genus plant extract that can be orally ingested without being subjected to processing into a pharmaceutical preparation, a food product, or the like, can be obtained.

After the purified Salacia genus plant extract (a purified essence) is obtained through the above-described extraction step, the essence may be subjected to centrifugal separation, filtration, and concentration to obtain a concentrated purified essence or subjected to drying to obtain a dried purified essence powder. The concentration or drying method is not particularly limited, and known methods may be used.

[Purified Salacia Genus Plant Extract]

In the purified Salacia genus plant extract according to the embodiment of the present invention, the content of epicatechin and the content of epigallocatechin are each less than 0.001% by mass with respect to the total amount of the purified Salacia genus plant extract. In the purified Salacia genus plant extract according to the embodiment of the present invention, the “purification” means to carry out purification so that the content of epicatechin and the content of epigallocatechin are each less than 0.001% by mass with respect to the total amount of the purified Salacia genus plant extract.

Since the purified Salacia genus plant extract according to the embodiment of the present invention has reduced bitter taste, it is an extract that can be orally ingested without being subjected to processing into a pharmaceutical preparation, a food product, or the like. Specifically, it has reduced bitter taste, the bitter taste being peculiar to Salacia genus plants, and it has characteristics described below.

(Component)

The bitterness of the purified Salacia genus plant extract can be judged by using the contents of epicatechin and epigallocatechin among the polyphenols as an indicator. From the viewpoint of further reducing bitter taste, the contents of epicatechin and epigallocatechin with respect to the total amount of the purified Salacia genus plant extract is less than 0.001% by mass.

Preferably, the content of mangiferin is less than 0.001% by mass with respect to the total amount of the purified Salacia genus plant extract.

(Content of Salacinol)

The content of salacinol in the purified Salacia genus plant extract according to the embodiment of the present invention can be checked by carrying out detection with high performance liquid chromatography under the following conditions.

Column: Shodex Asahipak NH2P-50 4E

Flow rate: 1 mL/minute

Eluent: 80% acetonitrile

Oven temperature: 30° C.

Injection amount: 25 μL

Detector: Charged aerosol detector (CAD)

(Evaluation Results for Flavor by Taste Sensor)

The flavor of the purified Salacia genus plant extract according to the embodiment of the present invention can be evaluated using a taste sensor. The taste sensor is made to imitate a human taste detection system, and it measures and evaluates the responsiveness to a taste substance toward a sensor using an artificial lipid membrane.

In the artificial lipid membrane used in the sensor of a taste sensor, since the responding taste substance varies depending on the type of lipid, the mixing ratio of a plasticizer, and the like, different responsiveness to the five basic tastes such as sour taste, salty taste, sweet taste, bitter taste, and delicious taste is exhibited by changing the type of the artificial lipid membrane. By utilizing this property, taste detection corresponding to each taste can be carried out, and in order to distinguish the foretaste and the aftertaste, the taste sensor can digitize and express taste through a plurality of items.

The artificial lipid membrane is stuck to the taste sensor surface, and as this membrane is immersed in a sample solution, there occurs a change in the membrane potential of the lipid membrane. As such, the amount of change in the membrane potential occurring in a case where the taste substance included in a sample solution adsorbs to the sensor surface is treated as the sensor output value, and thereby the taste of the measurement sample can be judged comprehensively.

Regarding the measurement, first, a taste sensor is immersed into a solution that serves as a reference (hereinafter, also referred to as “reference solution”), and the membrane potential Vr is obtained. Next, as the taste sensor is immersed in a sample solution, the membrane potential Vs, which has changed as a result of an interaction with a taste substance, is obtained. From this difference (Vs−Vr), a relative value of the sensor output is calculated, and this is a value corresponding to the foretaste.

Regarding the purified Salacia genus plant extract according to the embodiment of the present invention, in a case where a taste sensor using an artificial lipid membrane is immersed in a 0.3 mmol/L tartaric acid solution containing 30 mmol/L potassium chloride to acquire a membrane potential Vr, and the taste sensor is immersed in a sample solution containing the purified Salacia genus plant extract to acquire a membrane potential Vs, a foretaste that is a relative value calculated from Vs−Vr is preferably 7.00 or less, more preferably 6.00 or less, and still more preferably 5.00 or less.

Regarding the aftertaste, after the foretaste is measured by the method described above, the taste sensor is simply prewashed with the reference solution and is immersed again in the reference solution, and the membrane potential Vr′ is obtained. Thereby, from this membrane potential change (Vr′−Vr), the aftertaste can be determined as a CPA (Change of membrane Potential by Adsorption) value. The results obtained at the time of using a 0.3 mmol/L tartaric acid solution containing 30 mmol/L potassium chloride as a reference solution are used as the measured values.

Regarding the purified Salacia genus plant extract according to the embodiment of the present invention, in a case where a taste sensor using an artificial lipid membrane is immersed in a 0.3 mmol/L tartaric acid solution containing 30 mmol/L potassium chloride to acquire a membrane potential Vr, the taste sensor is immersed in a sample solution containing the purified Salacia genus plant extract to acquire a membrane potential Vs, the taste sensor is washed with a 0.3 mmol/L tartaric acid solution containing 30 mmol/L potassium chloride, and the taste sensor is immersed again in a 0.3 mmol/L tartaric acid solution containing 30 mmol/L potassium chloride to acquire a membrane potential Vr′, an aftertaste is preferably 0.5 or less, more preferably 0.3 or less, and still more preferably 0.1 or less, where the aftertaste is determined, as a value of change of membrane potential by adsorption, from a change in membrane potential which is the change represented by Vr′−Vr.

In the present invention, the foretaste is the taste felt immediately after a food product is taken into the mouth, and in a case where the foretaste is evaluated as a taste item by the above-described measurement, the foretaste is expressed as “sour taste”, “bitter taste”, “astringent taste”, “delicious taste”, and “salty taste”. On the other hand, the aftertaste is a taste remaining on the tongue even after the food product is swallowed, and in a case where the aftertaste is evaluated as a taste item by the above-described measurement, the aftertaste is expressed as “bitter taste”, “astringent taste”, or “delicious taste”.

The taste sensor that can be used in the present invention is not particularly limited; however, it is preferable to use a taste sensor having high responsiveness to the above-described five basic tastes, and above all, it is more preferable to use a taste sensor having high responsiveness to sour taste, salty taste, bitter taste, delicious taste, and astringent taste, from the viewpoint of analyzing the causes of the odor and bitter taste peculiar to Salacia genus plants, which significantly affect the flavor. Furthermore, the taste items obtained as the foretaste and/or aftertaste described above can also be used for the evaluation.

Regarding a commercially available measuring apparatus, for example, a taste perception apparatus, TS-5000Z (Intelligent Sensor Technology, Inc.), may be mentioned.

The purified Salacia genus plant extract according to the embodiment of the present invention can be preferably obtained by a method including an extraction step of passing a Salacia genus plant-containing raw material extract containing at least one of a Salacia genus plant, a Salacia genus plant extract, or a Salacia genus plant ground product, through a column packed with a styrene-based synthetic resin.

[Use Application]

The use application of the purified Salacia genus plant extract according to the embodiment of the present invention is not particularly limited, and examples thereof include food products (including beverages and supplements), food product materials, quasi-drugs, pharmaceutical products, pharmaceutical product materials, and quasi-drug materials. Specific examples thereof include those described in paragraphs 0031 to 0060 of JP2017-132763A. However, in a case where the purified Salacia genus plant extract of the present disclosure is applied to a food product, a food product material, a quasi-drug which is for use application on oral ingestion, a pharmaceutical product, a pharmaceutical material, and a quasi-drug material, the effect of the present invention is exhibited more effectively.

The present invention will be further described with reference to Examples below; however, the present invention is not limited to Examples below.

### EXAMPLES

### Example 1

(1) Preparation of Salacia Genus Plant-Containing Raw Material Extract

The root parts of Salacia reticulata were ground and then extracted with hot water, a solution thus obtained was subjected to centrifugal separation (rotation speed: 1,000 rpm, time: 10 minutes), and then the supernatant of the mixed solution was recovered, filtered, and cooled to room temperature to obtain a Salacia genus plant-containing raw material extract.

(2) Production of Purified Salacia Genus Plant Extract

The Salacia genus plant-containing raw material extract prepared in the procedure of the above (1) was passed through a column (ratio of diameter to height=1:3) packed with a styrene-divinylbenzene copolymer resin (specific surface area: 650 m²/g) at a flow rate of SV=1 to recover a treated liquid (the extraction step). The extract liquid (the treated liquid) was subjected to filtration, concentration, and spray drying to obtain a purified Salacia genus plant extract.

### Comparative Example 1

A purified Salacia genus plant extract was obtained by the same procedure except that the recovery of the treated liquid (the extraction step) by passing it through a column in the (2) of Example 1 was not carried out.

### Reference Example 1

(1) Preparation of Salacia Genus Plant-Containing Raw Material Extract

The root parts of Salacia reticulata were ground and then extracted with hot water, a solution thus obtained was concentrated, and a Salacia genus plant-containing raw material extract (solid content concentration: 10% to 15% by mass) was obtained.

(2) Production of Purified Salacia Genus Plant Extract

1.0% by mass of the activated carbon having an average pore diameter of 4.5 nm and a specific surface area of 1,700 m²/g was added with stirring to the Salacia genus plant-containing raw material extract prepared in the procedure of the above (1), thereby obtaining a mixed solution. The mixed solution thus obtained was heated to 50° C. while being stirred, and an extraction step was carried out by causing the mixed solution to react with the activated carbon for 60 minutes under stirring.

After the reaction, filtration and centrifugal separation (rotation speed: 1,050 rpm, time: 10 minutes) were carried out, and the supernatant of the mixed solution was recovered. Subsequently, the supernatant was subjected to concentration and spray drying, and thus a purified Salacia genus plant extract was obtained.

[Evaluation]

The purified Salacia genus plant extracts of Example 1, Comparative Example 1, and Reference Example 1, as well as Salacia genus plant extracts of commercial products A to C were evaluated as follows. The results are presented in the following tables.

(1) Salacinol Concentration

The salacinol concentration was measured according to the above-described experimental method. Subsequently, the salacinol concentration was calculated while the salacinol concentration of Comparative Example 1 was set to 100% by mass.

(2) Salacinol Recovery Rate

The salacinol recovery rate was calculated based on the following expression.

Salacinol recovery rate (%)=Salacia genus plant extract recovery rate (%)×salacinol concentration

(3) Measurement of Contents (in Terms of % by Mass) of Epicatechin (EC), Epigallocatechin (EGC), and Mangiferin

The contents of epicatechin, epigallocatechin, and mangiferin in the Salacia genus plant extract were quantified by detecting them with high performance liquid chromatography under the following conditions.

Column: LiChrosorb RB-18

Flow rate: 1 mL/minute

Eluent:

Liquid A, 0.05 mol/L phosphoric acid

Liquid B, a 0.05 mol/L phosphoric acid solution containing 40% acetonitrile

Gradient: A:B=80:20→(subsequently, from 10 to 60 minutes) 30:70

Oven temperature: 30° C.

Injection amount: 5 μL

Conditions for detector: Absorbance at 280 nm

(4) Evaluation of Bitter Taste or Astringent Taste

According to the experimental method described above in the present specification, the foretaste as “bitter taste” as well as the aftertaste as “bitter taste” or “astringent taste” was measured with a taste sensor.

A measuring solution was prepared so that the salacinol concentration was 1 mg/100 mL.

TABLE 1

| | Salacinol concentration | |
|---|---|---|
| | (% by mass) | Salacinol recovery rate |
| Example 1 | 164 | 100% |
| Comparative | 100 | 100% |
| Example 1 | | |
| Reference Example 1 | 133 | 97% |

TABLE 2

| | EC | EGC | Mangiferin |
|---|---|---|---|
| Example 1 | <0.001% | <0.001% | <0.001% |
| Comparative | 0.009% | 0.007% | 0.146% |
| Example 1 | | | |
| Reference | 0.002% | 0.001% | 0.034% |
| Example 1 | | | |
| Commercial | 0.003% | 0.005% | 0.428% |
| product A | | | |
| Commercial | 0.015% | 0.041% | 2.206% |
| product B | | | |
| Commercial | 0.031% | 0.118% | 1.139% |
| product C | | | |

TABLE 3

| | | | Astringent |
|---|---|---|---|
| | Bitter tase | | taste |
| | Aftertaste | Foretaste | Aftertaste |
| Example 1 | −0.06 | 4.59 | 0.54 |
| Comparative | 3.9 | 18.03 | 0.97 |
| Example 1 | | | |
| Reference | 1.2 | 8.89 | 0.65 |
| Example 1 | | | |
| Commercial | 1.66 | 9.51 | 0.87 |
| product A | | | |

From the above results, Example 1 showed a high recovery rate of salacinol, and furthermore, the salacinol concentration was high as compared with Comparative Example 1. In addition, the contents of epicatechin and epigallocatechin in Example 1 were extremely low as compared with Comparative Example 1. Regarding bitter taste and/or astringent taste as well, both the aftertaste and the foretaste were extremely low in Example 1 as compared with Comparative Example 1.

The purified Salacia genus plant extract according to the embodiment of the present invention can be applied to food products for the intended purpose of maintaining health.

What is claimed is:

1. A purified Salacia genus plant extract,
wherein a content of epicatechin and a content of epigallocatechin are each less than 0.001% by mass with respect to a total amount of the purified Salacia genus plant extract.

2. The purified Salacia genus plant extract according to claim 1,
wherein a content of mangiferin is less than 0.001% by mass with respect to the total amount of the purified Salacia genus plant extract.

3. The purified Salacia genus plant extract according to claim 1,
wherein in a case where a taste sensor that uses an artificial lipid membrane responding to bitter taste is immersed in a 0.3 mmol/L tartaric acid solution containing 30 mmol/L potassium chloride to acquire a membrane potential Vr, and the taste sensor is immersed in a sample solution containing the purified Salacia genus plant extract to acquire a membrane potential Vs, a foretaste that is a value calculated from Vs−Vr is 7.00 or less.

4. The purified Salacia genus plant extract according to claim 1,
wherein in a case where a taste sensor that uses an artificial lipid membrane responding to bitter taste or astringent taste is immersed in a 0.3 mmol/L tartaric acid solution containing 30 mmol/L potassium chloride to acquire a membrane potential Vr, the taste sensor is immersed in a sample solution containing the purified Salacia genus plant extract to acquire a membrane potential Vs, the taste sensor is washed with a 0.3 mmol/L tartaric acid solution containing 30 mmol/L potassium chloride, and the taste sensor is immersed again in a 0.3 mmol/L tartaric acid solution containing 30 mmol/L potassium chloride to acquire a membrane potential Vr′, an aftertaste is 0.6 or less, where the aftertaste is determined, as a value of change of membrane potential by adsorption, from a change in membrane potential which is the change represented by Vr′−Vr.

5. The purified Salacia genus plant extract according to claim 1,
wherein the purified Salacia genus plant extract is obtained by a method including an extraction step of passing a Salacia genus plant-containing raw material extract containing at least one of a Salacia genus plant, a Salacia genus plant extract, or a Salacia genus plant ground product, through a column packed with a styrene-based synthetic resin.

6. A production method for the purified Salacia genus plant extract according to claim 1, the production method comprising:
an extraction step of passing a Salacia genus plant-containing raw material extract containing at least one of a Salacia genus plant, a Salacia genus plant extract, or a Salacia genus plant ground product, through a column packed with a styrene-based synthetic resin.

7. The production method for a purified Salacia genus plant extract according to claim 6,
wherein the extraction step is carried out at a flow rate of SV=0.25 to 50 for liquid passage.

8. The production method for a purified Salacia genus plant extract according to claim 6,
wherein a ratio of a diameter to a height in the column is 1:1 to 1:12.

9. The production method for a purified Salacia genus plant extract according to claim 6,
wherein the styrene-based synthetic resin has a specific surface area of 300 to 1,500 m²/g.
